(19) United States
(12) Patent Application Publication
(10) Pub. No.: US 2007/0021699 A1
(43) Pub. Date: Jan. 25, 2007

(54) Orthopedic support devices with one or more lower modulus transition cuffs

(75) Inventors: Sam Braunstein, New York, NY (US); Jeffrey Stearns, Hopatcong, NJ (US)

Correspondence Address: WALL MARJAMA & BILINSKI, 101 SOUTH SALINA STREET, SUITE 400, SYRACUSE, NY, 13202, US

(73) Assignee: Wellgate Products, LLC, New York, NY (US)

(21) Appl. No.: 11/503,801

(22) Filed: Aug. 14, 2006

Related U.S. Application Data
(63) Continuation-in-part of application No. 11/185,333, filed on Jul. 20, 2005.

(51) Int. Cl. A61F 5/00

(52) U.S. Cl. 602/5

(57) ABSTRACT

Orthopedic support devices are provided that include a main body having at least one border area and causing targeted compression to a body part over which an orthopedic support device is worn and that further include at least one transition cuff located at a border area of the main body, wherein the main body is made of a material that has a predetermined elastic modulus and wherein the at least one transition cuff is made of a material that has a lower predetermined elastic modulus than the material from which the main body is made such that the presence of the at least one transition cuff is effective to reduce displacement of soft tissue at the border area that is caused by the targeted compression of the main body.

### CROSS REFERENCE TO RELATED APPLICATON

This application claims priority as a continuation-in-part application from, and incorporates by reference the entirety of currently pending U.S. patent application Ser. No. 11/185,333, which was filed on Jul. 20, 2005.

### FIELD OF THE INVENTION

The described embodiments relate to orthopedic support devices, and, in particular, to such devices that include one or more transition cuffs designed to minimize soft tissue displacement without detracting from the overall ability of the device to provide compression, protection and/or suspension to a body part.

### BACKGROUND OF THE INVENTION

Many people develop injuries in an area of their body (e.g., knee, ankle, elbow, wrist) that is utilized on a daily basis such that the injured area cannot be immobilized while the injury heals. Thus, the goal becomes to stabilize and protect the injured body part to an extent whereby some usage of the body part can occur while still allowing for there to be simultaneous healing. To that end, orthopedic support devices have been developed consisting of a layer of flexible, resilient material (e.g., neoprene) which, when stretched over a body part, provides support thereto.

Various problems have been observed with regard to these traditional orthopedic devices. For example, resilient materials neither effectively dissipate heat nor absorb/wick perspiration away from the skin. Thus, those who wear devices formed of such materials in warm climates and/or while engaged in strenuous physical activity may develop skin irritation, abrasions, heat rashes and/or dermatitis due to perspiration, particularly at points of bending such as the back, the knee, the elbow or the wrist.

Moreover, conventional resilient material orthopedic supports tend to migrate from their desired area of coverage, again owing to perspiration. Migration leaves the injured area entirely or partially unsupported, which, in turn, can result in slowed healing or even aggravation of the underlying injury. In a similar vein, resilient orthopedic supports have been known to sag, lose their shape or “bunch up,” e.g., when the supported body part is flexed. Bunching also can leave injured areas unprotected or only partially protected, and can either expedite the onset of skin problems already associated with such devices or create still other skin problems such as chafing or bruising.

Another common problem that has been observed with regard to traditional orthopedic support devices is what is generally referred to as soft tissue displacement. In order to provide support to an injured body part, an orthopedic support device applies compressive force thereto. As this occurs, however, the soft tissue under the device is displaced toward the nearest area(s) of non-compression, and, consequently, portions of the displaced soft tissue appear at or emerge from one or more border areas of the main body of the device. FIGS. 11A and 12A depict, respectively, two conventional orthopedic support devices 800, 900 being worn and in which displacement of soft tissue has occurred, wherein the areas of displaced soft tissue are referred to in the Figures with reference numeral 950.

Soft tissue displacement causes both functional and aesthetic problems for a wearer of an orthopedic support device. For one, it creates a loss of compression at the border areas where soft tissue displacement occurs. That, in turn, can cause a wearer of an affected orthopedic support device to develop or to be more likely to develop skin irritation, abrasions, bruising, and/or dermatitis while the device is being worn. These problems are even more likely to be experienced, or to be experienced even more severely, while the wearer is engaged in strenuous physical activity that involves the injured body part on or over which the orthopedic support device is being worn.

As is clearly illustrated in each of FIGS. 11A and 12A, soft tissue displacement often can actually create a bulging of the soft tissue. When severe, this bulging can limit the full range of motion of the wearer. For example, the presence of the bulged area of soft tissue 950 in FIG. 11A would likely cause a wearer of the device 800 to be unable to fully bend his or her knee while the device is being worn as intended.

Moreover, this bulging can be quite unsightly even if the wearer of the orthopedic support device is not heavy set, but especially if the wearer has excess soft tissue and/or cellulite at the bulging location(s). This is highly problematic because physical appearance is quite important to some people, often so much so that the unsightly bulging that necessarily occurs when wearing certain conventional orthopedic support devices (such as the devices 800, 900 of FIGS. 11A and 12A) might dissuade those who most need to wear such devices from actually wearing them.

Another issue arises due to the fact that many in the art attach higher modulus material (e.g., as a binding) to one or more border areas at the top, bottom, edges and/or openings of orthopedic support devices. This is done in order to increase the efficacy of the orthopedic support device by increasing its compressive force. However, the added compressive force creates even more soft tissue displacement than would normally occur at or near such border areas, and, in turn, can exacerbate the problems discussed above.

Therefore, a need exists for orthopedic support devices that avoid or minimize the litany of problems of conventional such devices, including but not limited to the particular problems of and associated with soft tissue displacement, yet that still can promote healing and enable freedom of movement while being worn.

### SUMMARY OF THE INVENTION

These and other needs are met by an orthopedic support device, which, by way of non-limiting example, can be a sleeve type or wrap type device. In an exemplary aspect, the orthopedic support device comprises (a) a main body formed of a first material that has a predetermined elastic modulus and that includes at least one border area, wherein the main body (which, if desired, can have a uniform patterned shape of substantially constant thickness) provides targeted compression and a substantially free range of movement to a body part when the orthopedic support device is worn so as to support the body part, and (b) at least one transition cuff which is attached to the main body and has a predetermined length (e.g., about 1 inch) and a predetermined elastic modulus, wherein its predetermined elastic modulus is lower than the predetermined elastic modulus of the main body, and wherein the presence of the at least one transition cuff is effective to reduce displacement of soft tissue at the at least one border area caused by the targeted compression of the main body.

In accordance with this, and, if desired, other exemplary aspects of the orthopedic support device, the equation:
0.15(X)≦Y≦0.80(X)
can be satisfied when the predetermined elastic modulus of the main body is X and the predetermined elastic modulus of the at least one transition cuff is Y.

Also in accordance with this, and, if desired, other exemplary aspects of the orthopedic support device, the device includes at least one of a proximal and a distal transition cuff. If the device includes both a proximal transition cuff and a distal transition cuff, then the proximal transition cuff can have a first elastic modulus and the distal transition cuff can have a second elastic modulus that is different than the first elastic modulus. In further accordance with this, and, if desired, other exemplary aspects of the orthopedic support device, the at least one transition cuff has a top side and an underside, wherein the at least one transition cuff can include at least one elastomeric attachment that is attached to the underside of the transition cuff.

In still further accordance with this, and, if desired, other exemplary aspects of the orthopedic support device, the main body of the device can be made of a material selected from the group consisting of: a thermoplastic rubber, an elastic knit, an elastic weave, a polyvinylchloride, a styrene, an acrylic rubber, a butadiene, a choloroprene, a chlorosulfonated elastomer, an ethylene copolymer, an ethylene vinyl acetate, a fluoro-rubber, a natural rubber, a nitrile elastomer, a hi-nitrile phenolic rubber, an epicholorohydrin, and a vinyl plasticol. Also, the at least one transition cuff can be made, for example, of a material selected from the group consisting of: an elastic spacer fabric and a spandex.

In accordance with an exemplary aspect of a wrap type orthopedic support device, the device can further comprise a first, proximal strap that extends outwardly from the main body and that has a first transition cuff attached thereto, and a second, distal strap extending outwardly from the main body and having a second transition cuff attached thereto. The first strap can extend outwardly from the main body in a different direction than the second strap and/or the first transition cuff can be located at or along a distal end of the first, proximal strap, and the second transition cuff can be located at or along a proximal end of the second, distal strap.

In accordance with another exemplary aspect, a sleeve type orthopedic device comprises (a) a main body that is formed of a resilient material, has a predetermined elastic modulus, and has at least one border area, where the main body provides targeted compression and a substantially free range of movement to a body part when the orthopedic support device is worn so as to support the body part, and (b) at least one transition cuff that is attached to the main body and that has a predetermined length, wherein the at least one transition cuff has a predetermined elastic modulus that is lower than the predetermined elastic modulus of the main body, and wherein the presence of the at least one transition cuff is effective to reduce displacement of soft tissue at the at least one border area that is caused by the targeted compression of the main body.

In accordance with yet another exemplary aspect, a support brace for the knee comprises a substantially tubular sleeve, wherein the sleeve itself comprises a main body that has a proximal end and a distal end and is made of a resilient material (e.g., a knitted elastic or a neoprene laminate), and a transition cuff that is located at the proximal end of the main body and that is made of an elastic spacer fabric, wherein the main body of the sleeve has a first predetermined elastic modulus and the transition cuff of the sleeve has a second predetermined elastic modulus, and wherein the second predetermined elastic modulus is equal to about 15% to about 80% of the first predetermined elastic modulus.

Still other aspects and advantages of these and other embodiments are discussed in detail below.

### BRIEF DESCRIPTION OF THE DRAWINGS

For a fuller understanding of the nature and desired objects of the aspects and embodiments described herein, reference is made to the following detailed description taken in conjunction with the accompanying figures, wherein like reference characters denote corresponding parts throughout the views, and in which:

FIG. 1 is front, perspective view of an orthopedic support sleeve of the present invention being worn on a knee;

FIG. 2 is a top view of the orthopedic support sleeve of FIG. 1 in an unworn condition;

FIG. 3 is a front, perspective view of an alternative embodiment of the orthopedic support sleeve of FIG. 1 being worn on a knee;

FIG. 4 is top view of a front panel of an orthopedic support sleeve of the present invention that includes a shaped buttress;

FIG. 5 is a side, sectional view of the front panel of FIG. 4 along the line 5-5;

FIG. 6 is an exploded view of the front panel of FIG. 4;

FIG. 7 is a top view of orthopedic patellar tendon bearing support device of the present invention;

FIG. 8 is a top view of the support buttress of the orthopedic device of FIG. 7

FIG. 9 is a bottom view of the support buttress of the orthopedic device of FIG. 7; and

FIG. 10 is a front view of the orthopedic support device of FIG. 7 as worn.

FIG. 11A is a posterior view of a conventional wrap-type orthopedic support device, which, as worn, causes soft tissue displacement;

FIG. 11B is a posterior view of an exemplary wrap-type orthopedic support device, which, as worn, reduces soft tissue displacement;

FIG. 11C is a top view of the orthopedic support device of FIG. 11B in an unworn, unwrapped condition;

FIG. 12A is an anterior view of a conventional sleeve-type orthopedic support device, which, as worn, causes soft tissue displacement;

FIG. 12B is an anterior view of an exemplary sleeve-type orthopedic support device, which, as worn, reduces soft tissue displacement;

FIG. 13 is a front view of a first exemplary orthopedic support device that includes a proximal transition cuff;

FIG. 14 is a side view of a second exemplary orthopedic support device that includes a proximal transition cuff; and

FIG. 15 is a posterior view of an exemplary lumbar support device that includes both a proximal transition cuff and a distal transition cuff.

### DETAILED DESCRIPTION

Exemplary embodiment of orthopedic support devices are provided in which a first material layer is attached directly onto, atop, or around a second material layer, wherein the first material layer provides support and/or compression, and the second material layer provides breathability and/or wicking. As will be described in detail below, and in accordance with such embodiments, certain characteristics (e.g., hardness, modulus of elasticity, shape, thickness, and/or location) of the first material layer can be varied to vary the location and/or the amount/level of compression provided by the support device. That, in turn, enables cost effective formation of a support device that can provide targeted compression to an injured body part with an optimal combination of healing and freedom of movement while the device is being worn.

In such embodiments, it is the first material layer that generally provides compression when the orthopedic device is worn. In an exemplary embodiment of the present invention, the first material layer is formed of one or more elastomeric or rubber materials. Suitable such materials include, but are not limited to materials having a hardness in the range of Shore 00-30 to Shore A-50 and/or a modulus of elasticity in the range of about 10 psi to about 150 psi at about 200% stretch, such as a thermoplastic rubber material (e.g., a thermoplastic elastomer), a silicone material (e.g., a silicone elastomer), an elastic knit or weave (e.g., a combination of a non-elastic fiber and an elastic material), a polyurethane material, a polyvinylchloride material, a styrene material (e.g., styrene-butadiene rubber, styrene-butadiene rubber and natural rubber blend), an acrylic rubber (e.g., polyacrylate, ethylene acrylic rubber), a polyester-urethane (e.g., ADIPRENE®), a butadiene-based material (e.g., polybutadiene, butyl rubber, hi-nitrile butadiene rubber), a choloroprene material (e.g., neoprene), a chlorosulfonated elastomer, an ethylene copolymer, an ethylene vinyl acetate material, a fluoro-rubber, a natural rubber, a nitrile elastomer, a hi-nitrile phenolic rubber, an epicholorohydrin material, and a vinyl plasticol material.

Generally, the material that forms the first material layer will have a higher modulus of elasticity than the material that forms the second material layer, so as to provide the orthopedic support with a desirable overall combination of support, compression and breathability, while still allowing for freedom of movement of the area on or over which the support is worn. However, it is understood that the material that forms the first material layer can have a modulus of elasticity less than or equal to the modulus of elasticity of the material that forms the second material layer without departing from the scope of the present invention.

The second material layer generally provides a buffer layer between the first material layer and the wearer's skin, but can have other functions and placements as well. According to an exemplary embodiment of the present invention, the second material layer is made of a material that is substantially breathable, has good wicking characteristics, and/or is resistant to bunching and migration when the orthopedic device is worn. Suitable such materials include, but are not limited to elastic spacer fabric made from nylon or polyester.

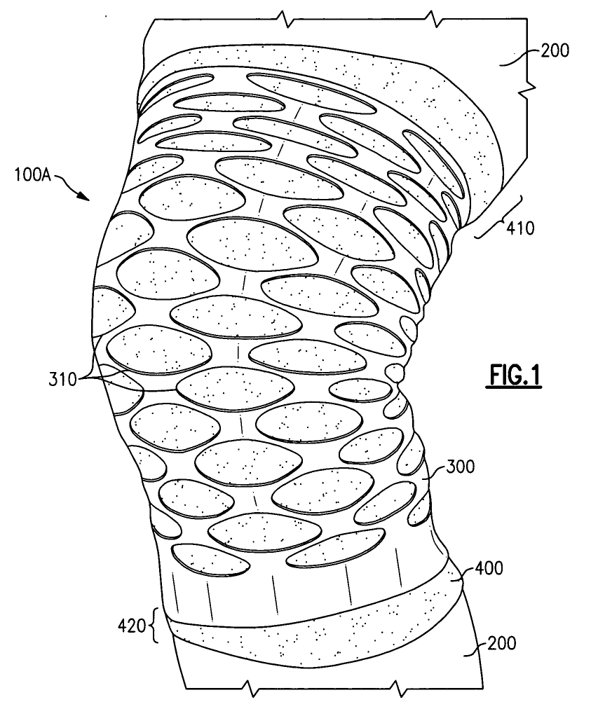

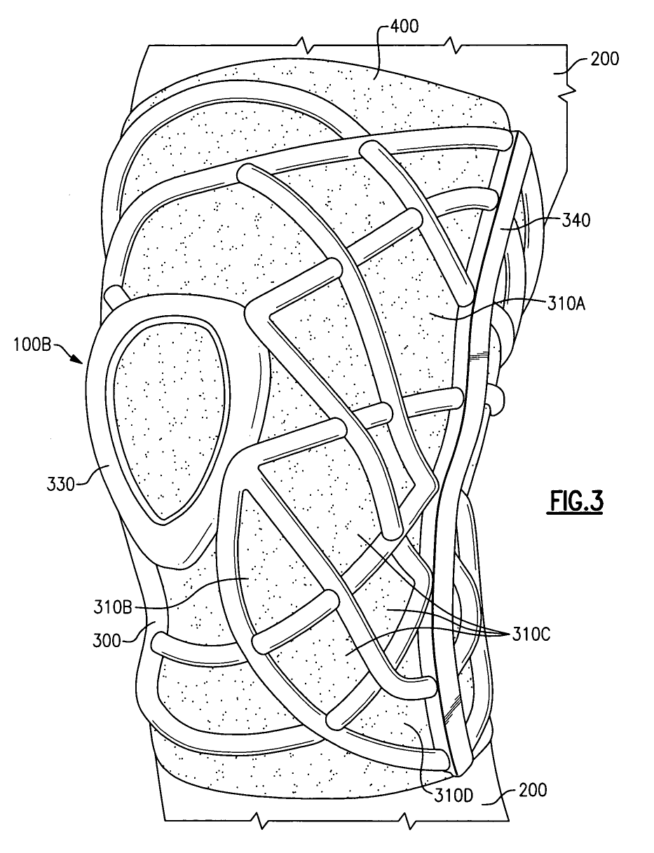

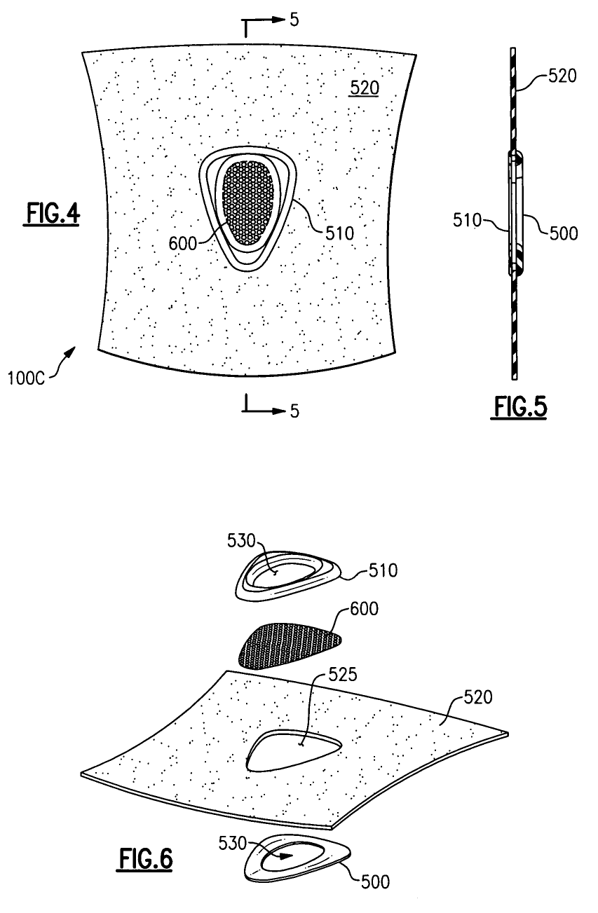

In accordance with various exemplary embodiments of the present invention, the first material layer can have a substantially constant or varied thickness, design and/or shape over some or all of its overall length. For example, and as will be discussed in greater detail below, the first material layer can have a uniform patterned shape of substantially constant thickness (see, e.g., the sleeve 100A illustrated in FIG. 1) or the first material layer can have a non-uniform patterned shape of non-constant thickness (see, e.g., the sleeve 100B illustrated in FIG. 3), and/or the first material layer can entirely comprise a shaped area of added thickness (see, e.g., the sleeve 100C depicted in FIGS. 4-6).

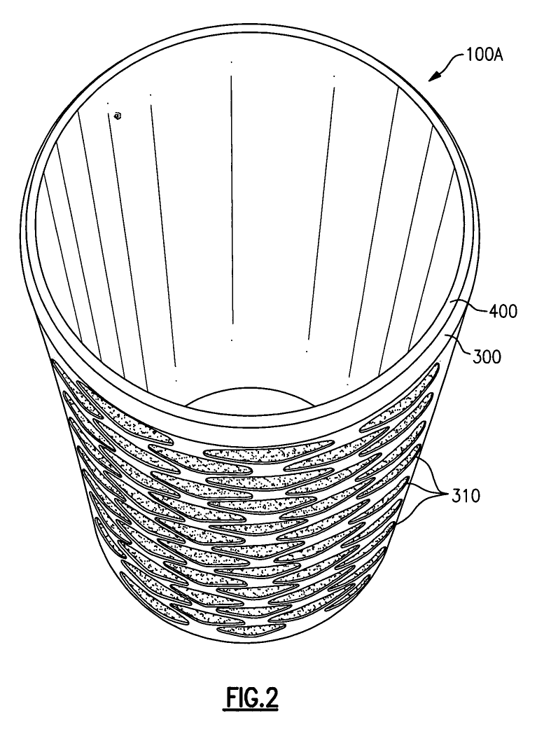

Referring initially to FIGS. 1 and 2, an exemplary embodiment of an orthopedic support device 100A according to the present invention is shown. In the depicted embodiment, the support device 100A is a sleeve, which, as shown in FIG. 1, can be worn over a portion of a wearer's leg 200 such that the first material layer 300 provides a compressive force on and around the wearer's knee and such that the second material layer 400 is in direct contact with the wearer's skin, thus acting as a barrier layer between the first material layer and the wearer.

The overall length of the second material layer 400 of the sleeve 100A can be less than, substantially equal to or greater than the overall length of the first material layer 300 of the sleeve 100A. As depicted in FIG. 1, and in accordance with an exemplary embodiment of the present invention, the length of the second material layer 400 of the sleeve 100A is greater than the length of the first material layer 300 such that one or more portions 410, 420 of the second material layer 400 extend above and/or below the first material layer 300, thus enabling the sleeve 100A to serve as a lower modulus control top in order to minimize displacement of soft tissue.

As best illustrated in FIG. 2, the first and second material layers 300, 400 are present around the entire circumference of the sleeve 100A in accordance with this exemplary embodiment of the present invention. FIG. 2 also illustrates that the first material layer 300 is formed directly atop/around and not encapsulated within the second material layer 400; however, it is understood that can be at least partially encapsulated within the second material layer 400 if instead desired. Also, although FIGS. 1 and 2 depict the first material layer 300 being present around the entire sleeve 100A (i.e., having 360° of coverage), it should be noted that the amount of coverage of the first material layer 300 can be less than about 360° in accordance with the present invention, e.g., such that when the sleeve device 100A is worn the first material layer can correspond to the front, back or side of a knee.

FIGS. 1 and 2 depict an exemplary embodiment of a sleeve 100A wherein the first material layer 300 has a substantially repeating, patterned shape, which, in this exemplary embodiment, resembles a web, but which can take on other shapes as well. To form the FIG. 1 pattern, the first material layer 300 of the sleeve 100A has a plurality of linked circular or elliptical openings 310 defined therein. The presence of these openings 310 is beneficial, since they better enable dissipation of heat and/or moisture (i.e., heat and/or moisture that has accumulated between the wearer's skin and the second material 400) through the breathable second material layer 400, thus reducing the occurrence, or at least the severity of skin problems associated with heat and/or moisture retention between the sleeve 100A and the wearer's skin. Moreover, the web-like pattern of openings 310 within the first material layer 300 provides an improved fit as well as increased comfort and an increased range of motion to the wearer, while still providing necessary targeted compression along substantially the entire sleeve 100A.

In an exemplary embodiment of the present invention wherein the sleeve 100A is designed for placement over a knee, the diameter of each opening 310 is generally in the range of about 0.25 inch to about 1.25 inch, and the total number of openings is generally in the range of about 100 about 200, and the distance between each opening is generally in the range of about 0.15 inch to about 0.80 inch. It is understood that the number, and/or the diameter, and/or the distance between one, some or all of the openings 310 within the first material layer of sleeve 100A can be modified within or outside these ranges for various reasons, e.g., to modify the level of compression sought to be provided by the sleeve 100A, to provide non-uniform compression at one or more areas, to adapt to an atypical anatomy of a wearer, to relieve compression at one or more pressure points, and/or to vary the range of motion provided to a wearer, and/or to improve the overall fit of the device. It is further understood that one or more portions of the first material layer 300 between and/or around the openings 310 can have various complex and/or compound radii and/or can have filleted areas and/or edges, thus allowing the modulus of elasticity of such portion(s) to be precisely tailored.

In accordance with an exemplary embodiment of the present invention in which the first material layer 300 has a plurality of openings 310 defined therein, at least a portion of the length of the first material layer does not include any openings in order to enhance the structural integrity of the device. By way of non-limiting example, such a portion can be located at or along the bottom and/or top end(s) of the first material layer 300 in order to prevent migration and bunching and to increase the durability of the sleeve 100A. Also by way of non-limiting example, and as shown in FIG. 1, such a portion can be located at or along the bottom end 320 of the first material layer 300 and generally will have a length in the range of about 0.10 inch to about 2.50 inch, wherein this length can be modified within or outside of this range for various reasons, e.g., to improve the overall fit of the device.

Because of the presence of the repeating pattern of openings 310 within its first material layer 300, the sleeve 100A of FIG. 1 provides substantially uniform, targeted compression and pressure when worn over an injured body part. It may be desired, however, to provide varied (i.e., non-uniform) compression through some portions of a sleeve 100A. For example, it may be desired to design the sleeve 100A to provide increased compression at the portion of the sleeve that will be worn directly over an injured area, e.g., a kneecap or a knee ligament. Such varied compression can be accomplished, e.g., by varying one or more of the shape of the openings 310 and/or by varying the thickness of the first material layer 300, and/or by varying the modulus of elasticity of the first material layer.

Referring now to FIG. 3, an alternate embodiment of the FIG. 1 sleeve 100A is shown that differs from the FIG. 1 sleeve in that its sleeve 100B provides a non-uniform level of compression by including a varied pattern of openings 310 and a varied thickness of the first material layer 300 in certain predetermined areas of the sleeve. Whereas the pattern of the first material layer 300 in the FIG. 1 sleeve 100A was substantially repeating, the pattern of the first material layer in the FIG. 3 sleeve 100B is substantially non-uniform (i.e., non-repeating). By way of non-limiting example, and as shown in the FIG. 3 exemplary embodiment, some openings 310A in the first material layer 300 of the sleeve 100B can have a pentagonal shape, while other openings 310B can have a triangular shape, and still other openings 310C can have a rhomboid shape, and yet still other openings 310D can have a trapezoidal shape. Each of these shapes represents not only a differently shaped opening, but also a somewhat differently sized opening; thus, in turn, each differently shaped opening provides a somewhat different level of compression beneath the opening. In general, the number of total openings 310A, 310B, 310C, 310D and the distance between each opening provided in the FIG. 3 sleeve 100B will be less than the total number of openings 310 and the distance between each opening in the FIG. 1 sleeve, whereas the size of the openings 310A, 310B, 310C, 310D in the FIG. 3 sleeve 100B generally will be greater than the size of the openings 310 in the FIG. 1 sleeve 100A.

Further, whereas the thickness of the first material layer 300 was substantially constant in the FIG. 1 sleeve 100A, one or more portions of the first material layer in the FIG. 3 sleeve 100B generally will have an increased thickness in order to provide one or more areas of increased, targeted compression. Also, the added thickness will prevent or at least further deter bunching and migration of the sleeve 100B. The number, location and/or shape of these increased thickness portions can vary; however, according to an exemplary embodiment of the present invention, the FIG. 3 sleeve includes at least two added thickness portions: a first added thickness portion 330 (i.e., a buttress) surrounding one of the circular openings 310 of the first material layer 300 that provides increased compression at a kneecap, and a second added thickness portion 340 that spans between the top and bottom ends of the first material layer to provide increased compression to the knee ligaments and to specifically deter migration and bunching of the sleeve 100B.

In the FIG. 1 embodiment 100A of the present invention, the thickness of the first material layer 300 is substantially constant and is generally in the range of about 0.05 inch to about 0.20 inch, and the thickness of the second material layer 400 is substantially constant and is generally in the range of about 0.05 inch to about 0.25 inch. In the FIG. 3 embodiment 100B, the thickness of the second material layer 400 is generally constant and within the same range as its thickness in the FIG. 1 embodiment and the thickness of the first material layer 300 is generally constant and within the same range as its thickness in the FIG. 1 embodiment, except at the increased thickness portions 330, 340 where the thickness of the first material layer is generally up to about 50% greater than that of the remainder of the first material layer. By way of non-limiting example, in a FIG. 3 embodiment wherein the thickness of the first material layer 300 is about 0.125 inch, the thickness of each of the increased thickness portions 330, 340 generally will be up to about 0.1875 inch. The hardness of the first material 300 generally will be about 20 Shore A in both the FIG. 1 embodiment 100A and the FIG. 2 embodiment 100B.

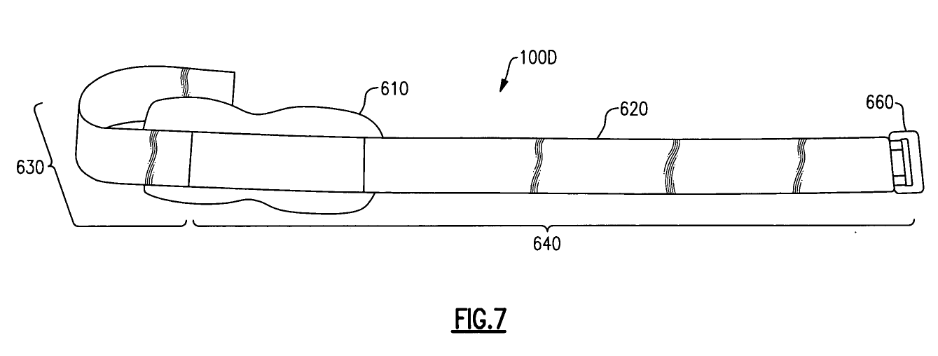

As noted above, the FIG. 3 sleeve 100B includes a buttress 330, which comprises a portion of the first material layer 300 and which has comparatively increased thickness to provide increased targeted compression. FIGS. 4-10 depict other exemplary embodiments of orthopedic devices 100C (see FIG. 4-6), 100D (see FIG. 7-10) in accordance with the present invention that also include one or more buttress portions formed at least partially of an elastomeric material.

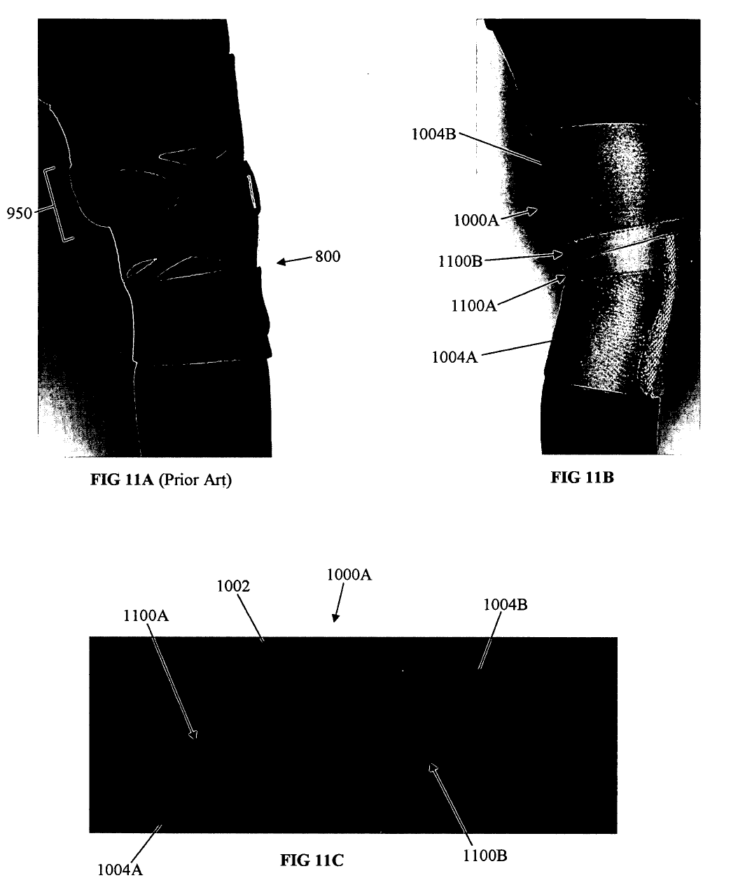

FIGS. 4-6 depict a top panel of an orthopedic device 100C. The device 100C can be a sleeve that is worn over a knee, in which case the top panel can be connected to a back panel (not shown) or can be fashioned into a sleeve, e.g., by extending the front panel to form a sleeve that would fit over a leg as do the sleeves 100A, 100B shown in FIGS. 1-3. Alternatively, the device 100C can be a wrap type device, such as is shown in FIG. 11C. The device 100C includes a panel/sleeve body 520 having an opening 525 (see FIG. 6), wherein a first buttress 500 is formed on the inside of the panel body and a second buttress 510 is formed on the outside of the panel body. According to an exemplary embodiment of the present invention, the first and second buttresses 500, 510 are located at the same position on the device 100C, wherein that position will correspond to a kneecap when the device is worn over a leg, and wherein the first, inner buttress 500 would be in direct contact with the kneecap. Each buttress 500, 510 generally includes an opening 530, wherein at least a portion of each buttress opening will overlap at least a portion of (optionally, substantially the entirety of) the opening 525.

Although two buttresses 500, 510 are illustrated in FIGS. 4-6, it should be noted that the FIG. 4 device 100C can include only one buttress or greater than two buttresses. In an embodiment wherein the device 100C includes only one buttress, the buttress generally would be located on the inside of the panel body 520, e.g., in a position that will correspond to a kneecap when the device is worn over a leg. Alternatively, however, a single buttress can be located on the outside of the panel body 520. In an embodiment wherein more than two buttresses are included, the additional buttress(es) can be located, e.g., inside and/or outside the sleeve 100C so as to correspond to positions at either or both sides of the knee in order to protect knee ligaments.

The first and second buttresses 500, 510 can have the same or different shapes and the same or different thicknesses. According to the exemplary embodiment of the FIGS. 4-6 sleeve 100C, the first and second buttresses 500, 510 have identical “shieldlike” shapes (i.e., resembling a rounded triangle) and each buttress has an elliptical opening 530 defined therein. Without wishing to be bound by theory, such a shape is believed to be well suited for providing targeted support and compression to a kneecap when the sleeve 100C is worn over a knee; however, other shapes for the buttresses 500, 510 and/or the openings 530 are within the scope of the present invention. The size of the openings 530 can vary, as can the size of the buttresses 500, 510 in which the opening is defined; however, according to an exemplary embodiment of the present invention, each opening covers about 35% to about 80% of the overall surface area of each buttress.

According to an exemplary embodiment of the present invention, and as best shown in FIG. 6, a fabric layer 600 can be provided between the inner buttress 500 and the outer buttress 510. The fabric layer 600 is selected to be highly breathable to allow air and/or moisture generated within the openings 530 to be breathed and/or wicked away from the skin. According to an exemplary embodiment of the present invention, the buttresses 500, 510 are made of an elastomeric material, the panel body 520 is made of a resilient material (e.g., neoprene) and the fabric layer 600 is made of mesh material.

Optionally, the device 100C can include first and second transition cuffs, which, if present, can be provided at a top portion and/or a bottom portion of the sleeve/panel body 520 such as described above with regard to the FIG. 1 sleeve 100A. According to an exemplary such embodiment, the one or more transition cuffs can be made of an elastic spacer fabric and would be present in order to minimize displacement of soft tissue.

The thickness and/or the hardness of each buttress 500, 510 can be identical or can vary. According to an exemplary embodiment of the present invention, the thickness of the first, inner buttress 500 is greater than the thickness of the second, outer buttress 510 and the hardness of the first, inner buttress is less than the hardness of the second, outer buttress. This enables the first, inner buttress 500 to provide cushioned support and the second, outer buttress 510 provides firmer, structural support.

The first, inner buttress 500 is generally about 1.5 to 3.5 times as thick (e.g., about twice as thick) as the second, outer buttress 510, wherein the thickness of the first, inner buttress 500 generally is in the range of about 0.15 inch to about 0.35 inch (e.g., about 0.25 inch) and the thickness of the second, outer buttress 510 generally is in the range of about 0.0425 inch to about 0.0825 inch (e.g., about 0.0625 inch). The hardness of the first, inner buttress 500 generally is about 40 Shore 00, and the hardness of the second, outer buttress 510 generally is about 40 Shore A.

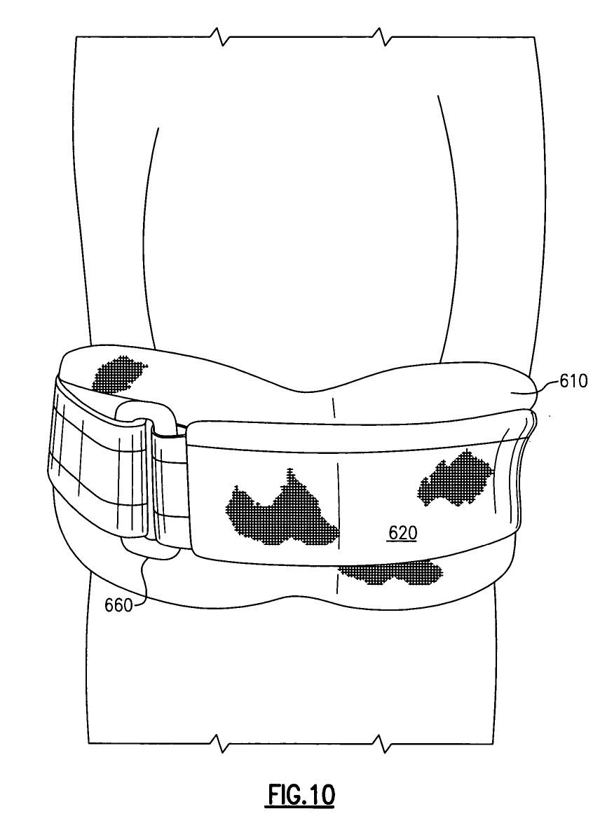

Another exemplary orthopedic support device 100D in accordance with the present invention is shown in FIGS. 7-10. This device 100D is patella tendon bearing device and includes a support buttress 610 having an attached strap 620, wherein the strap has a first strap portion 630 and a second strap portion 640, and wherein the second strap portion culminates in a D-ring or buckle 660 that is configured to accept the first strap portion 630. By way of non-limiting example, the first strap portion 630 can be made of hook material (e.g., velcro) and the second strap portion 640 can be made of loop material (e.g., velcro) to help maintain the device 100D in place when worn as shown in FIG. 10.

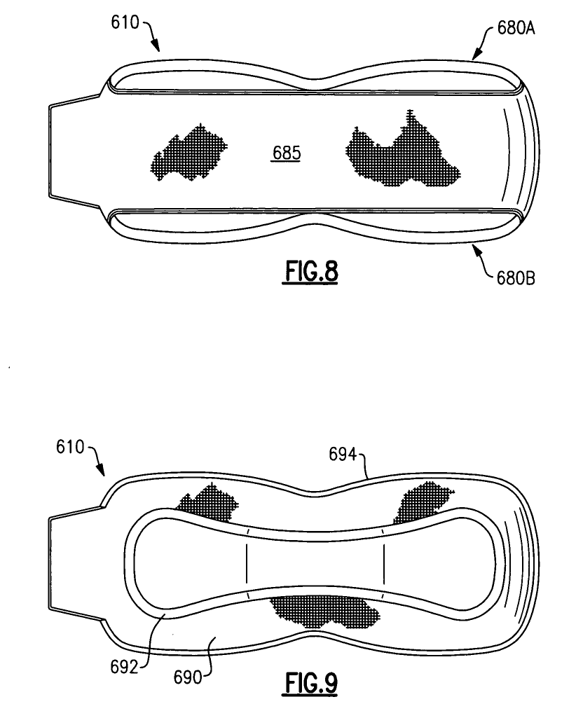

As best shown in FIG. 8, the top side of the support buttress 610 includes first and second raised portions 680A, 680B (e.g., rails) shaped to function as a strap guide, wherein a strap guide area 685 is defined between the first and second rails on which the strap portion 620 will rest when the device 100D is being worn (see FIG. 10). In accordance with an exemplary embodiment of the present invention, the first and second raised potions 680A, 680B of the support buttress 610 are made solely of an elastomeric material, and the strap guide area 685 between the first and second raised portions are made of a combination of an elastomeric material and a spacer fabric. Generally, the same elastomeric material is utilized to form both raised portions 680A, 680B of the buttress 610, wherein according to an exemplary embodiment of the present invention the hardness of the elastomeric material is about 10 Shore A to about 20 Shore A. The elastomeric material within the combination of materials utilized to form the strap guide area 685 can have the same or different hardness as the elastomeric material that comprises the raised portions 680A, 680B.

Support buttress 610 can have a variety of shapes; however, as shown in FIGS. 8 and 9, and according to an exemplary embodiment of the present invention, the support buttress has a concave shape resembling a bowtie or hourglass. When the device 100D is worn as shown in FIG. 10, the underside 690 (see FIG. 9) of support buttress 610 is in direct contact with the wearer's leg 200 and the strap 620 is wrapped around the support buttress such that the strap is in direction communication with the strap guide area 685 between the raised portions 680A, 680B of the support buttress.

In accordance with an exemplary embodiment of the present invention, and as best shown in FIG. 9, the inner segment 692 of the underside 690 of support buttress 610 protrudes from the underside by a predetermined height and is formed of an elastomeric material, whereas the outer segment 694 that surrounds the inner segment 692 is formed of a combination of elastomeric material and spacer fabric. Generally, the elastomeric material that forms the inner segment 692 of the underside 690 of the support buttress 610 has a lower hardness (e.g., 5 Shore A) than the hardness (e.g., 10 Shore A) of the elastomeric material that forms the outer segment 694.

Any of these various orthopedic support devices 100A-100D, and others, can be configured to include one or more transition cuffs. For example, in FIG. 1, the orthopedic support device 100A includes a main body 300 and an underlying layer 400 of material. The underlying layer 400 culminates in a first transition cuff 410—referred to above as a control top—located at or along the top (i.e., proximal end) of the device 100A at an area that forms a border between a wearer's skin and the main body 300 of the device, and a second transition cuff 420—also referred to above as a control top—located at or along the bottom (i.e., distal end) of the device at an area that also forms a border between the wearer's skin and the device. Each transition cuff extends beyond the main body by a predetermined length, e.g., about 1 inch. It should be noted that if instead desired, the underlying layer 400 can form only a proximal transition cuff 410 or only a distal transition cuff 420. Moreover, also if desired the length of either or each transition cuff 410, 420 can be greater or less than 1 inch. As explained in detail below, the presence of one or more transition cuffs beneficially reduces soft tissue displacement caused by an orthopedic support device, and thus, in turn, reduces or eliminates the functional and aesthetic problems associated with soft tissue displacement.

In the FIG. 1 exemplary embodiment, and in general, each transition cuff creates a buffer between the main body of an orthopedic support device and an top, bottom, edge, side, opening, perimeter or other border area of the device. Any orthopedic support device can include one or more transition cuffs, wherein the choice of how many to include and where to locate the transition cuff(s) is influenced by factors such as: the body part over which the device is intended to be worn, the likelihood of a wearer to suffer from soft tissue displacement, the design of the device, etc. For example, the problem of soft tissue displacement is likely to be more prevalent with regard to orthopedic support devices, which, when worn, have border areas that coincide with portions of a wearer's body that tend to have excess fatty soft tissue, such as the thigh, upper buttocks or abdomen. Thus, such orthopedic support devices may include more and/or differently configured transition cuffs than an orthopedic support device that is intended to be worn elsewhere (e.g., over the elbow).

In an embodiment in which an orthopedic support device includes one or more transition cuffs, it is currently preferred for each such cuff to have a comparatively lower elastic modulus than at least a portion, if not all of the main body of the device. The term elastic modulus, as used herein, is defined as the pounds per square inch (psi) of force required to stretch or elongate a material. It has been observed by those skilled in the art that an orthopedic support device generally should be capable of stretching by about 25% to about 35% from its unworn shape in order to fit over an injured body part yet also to provide the necessary compressive force to protect the injured body part when the device is being worn. This enables the transition cuff(s) to act as an elastic modulus transition from the higher modulus main body of the device to a border area of the device. Also, this is in contrast to conventional orthopedic support devices, which either have the same elastic modulus throughout the device or include a binding that has a higher modulus than the main body. However, it is also understood that certain orthopedic support devices can serve these purposes while being capable of stretching outside of the about 25% to about 35% range.

By having a comparatively lower elastic modulus than the main body, the one or more transition cuffs will produce a comparatively lower compressive force than the main body. As noted above, it is the high compressive force of the main body of an orthopedic support device that tends to cause soft tissue displacement. Thus, one or more transition cuffs can be beneficially placed at one or more border areas so as to minimize or reduce or curb the displacement of soft tissue. In addition, the presence of the one or more lower elastic modulus transition cuffs beneficially still allows the main body of the orthopedic support device to have a high enough modulus such that the main body can continue to apply the compressive force that provides the necessary support, protection and/or suspension to the injured body part over which the device is being worn.

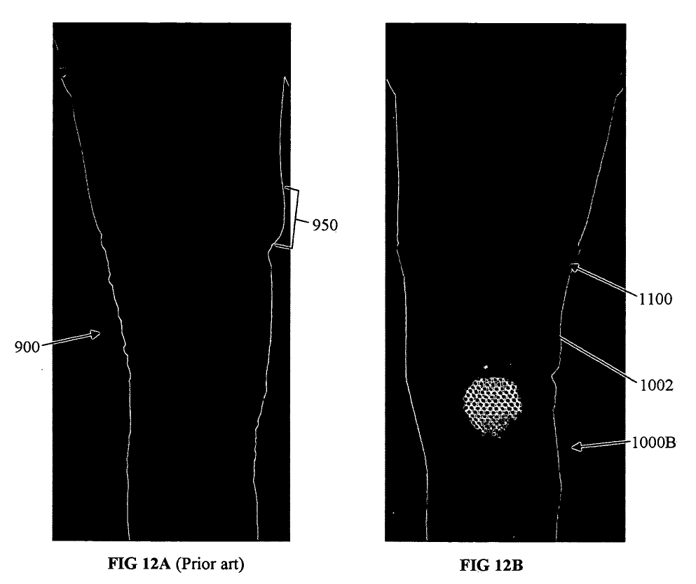

The beneficial effect of including one or more transition cuffs that have a lower modulus than the main body of an orthopedic support device is clearly shown by comparing FIG. 11A to FIG. 11B and FIG. 12A to FIG. 12B. FIG. 11A depicts a conventional wrap type orthopedic knee support device 800, which, as worn, causes visually apparent soft tissue displacement (see reference numeral 950). FIGS. 11B and 11C depict a wrap type orthopedic support device 1000A that includes a main body 1002, a proximal wing/flap 1004B and a distal wing/flap 1004A, wherein each flap includes a transition cuff 1100. By way of non-limiting example, and as shown in FIGS. 11B and 11C, the proximal flap 1004B can have a transition cuff 1100B that is located at or along the distal end of the proximal flap, whereas the distal flap 1004A can have a transition cuff 1100A that is located at or along the proximal end of the distal flap. Thus, as best shown by FIG. 11B, when the device 1000A is worn, the transition cuffs 1100A, 1100B are adjacent to each other and the device 1000A does not cause visually apparent soft tissue displacement. However, the device 1000A provides support, protection and suspension to an injured body part (e.g., knee) over which it was worn.

FIG. 12A depicts a conventional sleeve type orthopedic support device 900, which, when worn, causes soft tissue displacement—as denoted with reference numeral 950—at the proximal end of the device. Conversely, FIG. 12B depicts an exemplary substantially tubular sleeve type orthopedic support device 1000B that includes a main body 1002 and at least a proximal transition cuff 1100 and which, as worn (e.g., as a support brace over a knee), does not cause soft tissue displacement at its proximal end or elsewhere.

As noted above, each transition cuff 1100 generally has a lower elastic modulus than at least a portion, if not the entirety, of the main body 1002 of the orthopedic support device 1000 of which it is part. In accordance with an exemplary embodiment, the elastic modulus of each transition cuff 1100 can be in the range of about 15% to about 80% of the elastic modulus of the main body 1002. This range also encompasses any and all subranges therebetween. Thus, if the elastic modulus of the material from which the main body 1002 of an orthopedic support device 1000 is made is denoted as “X” and the elastic modulus of the material from which the one or more transition cuffs 1100 are made is denoted as “Y,” then Equation 1 (see below) is satisfied as follows:
0.15(X)≦Y≦0.80(X) [Equation 1]

It should be noted, however, that the specific percentages listed in Equation 1 can vary according to one or more factors such as the body part over which the device is intended to be worn, the likelihood of a wearer to suffer from soft tissue displacement, the design of the device, etc. Moreover, in accordance with an embodiment wherein a support device 1000 includes more than one transition cuff 1100, although Equation 1 generally is satisfied, each transition cuff can, but need not, have the same difference in elastic modulus from that of the main body 1002 of the device. In other words, if a first transition cuff has an elastic modulus equal to 45% of the elastic modulus of the main body material, then a second transition cuff can have an elastic modulus that is also equal 45% of the elastic modulus of the main body material, or, instead, the elastic modulus of the second transition cuff can fall within the parameters of Equation 1 but could be greater or less than 45% of the elastic modulus of the main body.

In order to satisfy this difference in elastic modulus between the transition cuff(s) 1100 and the main body 1002 of an orthopedic support device 1000 in accordance with Equation 1, it is currently preferred for the transition cuff(s) to be made of different materials than the main body. However, it is possible for the equation to be satisfied despite the transition cuff(s) 1100 and the main body 1002 being made of the same material. For example, the transition cuff(s) could satisfy Equation 1 by being made of a different grade, style, type and/or physical configuration of the same material as the main body.

By way of non-limiting example, suitable materials from which the main body 1002 of the device 1000 can be formed include, but are not limited to, resilient materials such thermoplastic rubber material (e.g., a thermoplastic elastomer), an elastic knit or weave (e.g., a combination of a non-elastic fiber and an elastic material), a silicone material (e.g., a silicone elastomer), a polyurethane material, a polyvinylchloride material, a styrene material (e.g., styrene-butadiene rubber, styrene-butadiene rubber and natural rubber blend), an acrylic rubber (e.g., polyacrylate, ethylene acrylic rubber), a polyester-urethane (e.g., ADIPRENE®), a butadiene-based material (e.g., polybutadiene, butyl rubber, hi-nitrile butadiene rubber), a choloroprene material (e.g., neoprene), a chlorosulfonated elastomer, an ethylene copolymer, an ethylene vinyl acetate material, a fluoro-rubber, a natural rubber, a nitrile elastomer, a hi-nitrile phenolic rubber, an epicholorohydrin material, and a vinyl plasticol material. When the main body 1002 of an orthopedic support device 1000 is formed of one of these materials, there are various materials from which the one or more transition cuff(s) 1100 can be formed that can satisfy Equation 1. Such materials for the transition cuff(s) 1100 include, but are not limited to, elastic spacer fabrics made of, e.g., nylon or polyester, or a polyurethane-containing material (e.g., spandex).

To demonstrate that such materials combinations can satisfy Equation 1, an exemplary experiment was performed wherein a piece of neoprene laminate that was about 12 inches long, about 3 inches wide, and about 0.08 inch thick and a piece of elastic spacer fabric having about the same dimensions were separately stretched in a lengthwise direction until each of their lengths was increased by about 25% (i.e., to about 15 inches in length). The neoprene laminate required about 0.066 psi of force to stretch to 15 inches in length, whereas the elastic spacer fabric required only about 0.024 psi to stretch to 15 inches in length. Thus, the elastic spacer fabric has an elastic modulus that is about 36% of the elastic modulus of neoprene laminate. Accordingly, Equation 1 is satisfied, for example, when a main body 1002 of an orthopedic support device 1000 is made of a neoprene laminate and wherein one or more transition cuffs 1100 is/are made of an elastic spacer fabric.

Despite having a comparatively lower elastic modulus than the main body 1002 of the orthopedic support device 1000, the one or more transition cuff(s) 1100 of the device have enough structural integrity (i.e., body) to stay in place while the device is being worn as intended. For example, the FIG. 12B device 1000B includes a proximal transition cuff 1100, which, if it lacked structural integrity, would fold over in a distal direction toward the main body 1002 of the device. If such folding over was to occur, the transition cuff 1100 would be less able—if not substantially unable—to serve its purpose of eliminating or reducing displacement of surrounding soft tissue.

At least certain materials (e.g., elastic spacer fabrics) which satisfy Equation 1 will have enough structural integrity to stay in place to a suitable degree such that neither soft tissue displacement nor migration will occur, even under strenuous conditions and in the presence of perspiration or other moisture. It should be noted, however, that certain other materials (e.g., spandex) which can satisfy Equation 1 may not have enough structural integrity to prevent the folding over problem from occurring with respect to a transition cuff from which the material is made. In such instances, and, if desired, in others, one or more elastomeric attachments can be attached or connected (e.g., via an adhesive or molding) to the underside (i.e., the side that is in contact with a wearer's skin) of the one or more transition cuff. The presence of the elastomeric attachment(s) helps maintain contact between the underside of the transition cuff and the wearer's skin, thus enabling the cuff to remain in place and, as such, to remain able to eliminate or reduce the problem of soft tissue displacement.

It should be noted that the length of this or any transition cuff 1100 can vary above or below the stated length in accordance with one or more factors, such as the elastic moduli of the various materials, the design of the device, the body part on which the device is intended to be worn, the likelihood of the wearer to suffer from soft tissue displacement, etc. Moreover, a transition cuff 1100 can be attached to the main body 1002 of an orthopedic support device 1000 via one of many techniques known in the art (e.g., sewing, use of an adhesive). A transition cuff 1100 can be attached such that the length of the transition cuff, as attached, is equal to the desired length of the transition cuff. Alternatively, a transition cuff 1100 can have a pre-attachment length of approximately twice that of the desired length of the transition cuff. In such instances, the transition cuff can be attached at one end, then folded in half lengthwise, and then attached at the other end.

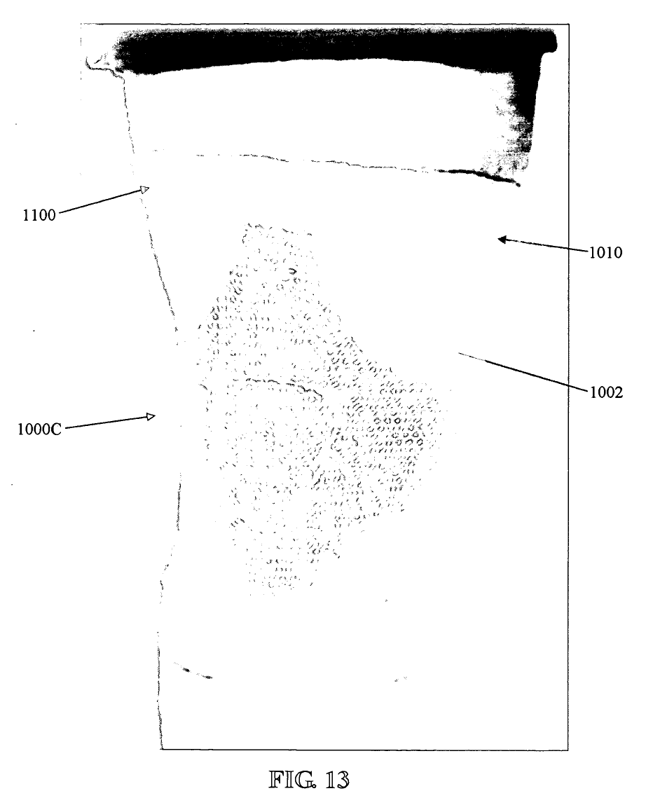

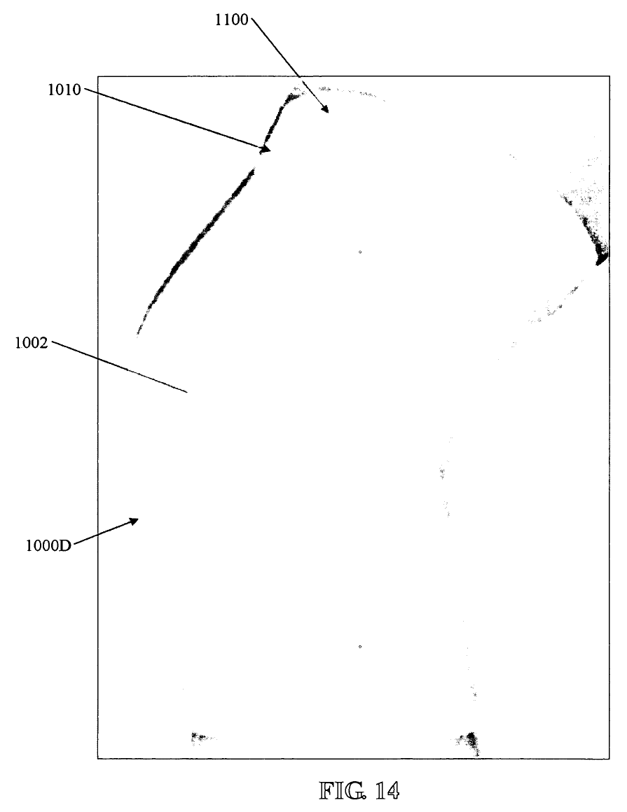

Referring now to FIGS. 13 and 14, two additional sleeve type orthopedic support sleeve devices 1000C, 1000D are shown being worn to protect a knee. In each instance the device 1000C, 1000D includes a transition cuff 1100 located at the proximal end 1010 of the device. The proximal transition cuff 1100 of each of the FIG. 13 device 1000C and the FIG. 14 1000D device has a length of about 1 inch—that is, each transition cuff extends in the proximal direction by about 1 inch in length from the proximal end 1010 of the main body 1002 of the device.

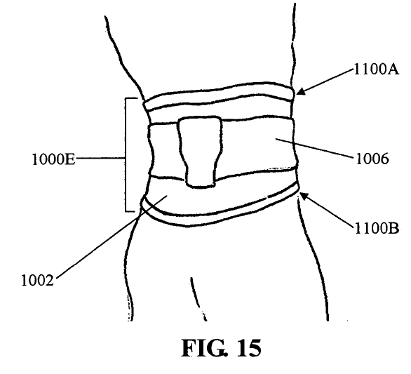

It is also possible to have a transition cuff located at each of the top (i.e., proximal) end and the bottom (i.e., distal) end of any of the aforementioned orthopedic support devices. Moreover, as shown in FIG. 15, an exemplary lumbar support device 1000E can include two transition cuffs. The FIG. 15 device 1000E includes a main body 1002, a strap area 1006, a proximal transition cuff 1100A and a distal transition cuff 1100B. When the orthopedic support device 1000E is worn as intended, and as is depicted in FIG. 15, the cuffs 1100A, 1100B are located at or near the abdomen and upper buttocks areas of a wearer, each of which is an area at which even those who are considered in shape can have a significant amount soft tissue that is capable of being displaced. Thus, the presence of the transition cuffs 1100A, 1100B eliminates or at least substantially curtails the problem of soft tissue displacement when the device 1000E is worn as intended.

The orthopedic support devices of the present invention can be formed by one or more techniques known in the art, including, but not limited to, a transfer molding process. Transfer molding is an advantageous manufacturing process in accordance with the present invention because it inexpensively allows a manufacturer to vary one or more of the size, shape, pattern, thickness and location of some or all of the materials that form the end product (i.e., the support device) in order to vary the level/amount and location of compression provided by the support device. Moreover, in further accordance with the present invention, a transfer molding process enables a first material layer to be deposited onto a second material layer or substrate such that some, none or all of the first material layer flows through or encapsulates the second material layer/substrate.

It is understood that although the exemplary embodiments depicted in the figures and described herein pertain either to knee, lumbar or patella support devices, wherein such devices in accordance with the present invention can be modified without departing from the scope of the present invention so as to be wearable to provide support to other injured body parts, e.g., an elbow, an arm, a leg, an ankle, the back, a shoulder, a wrist, the neck, that can become injured to an extent that the body part requires support or would benefit from being supported while the injured body part heals.

Although the present invention has been described herein with reference to details of currently preferred embodiments, it is not intended that such details be regarded as limiting the scope of the invention, except as and to the extent that they are included in the following claims—that is, the foregoing description of the present invention is merely illustrative, and it should be understood that variations and modifications can be effected without departing from the scope or spirit of the invention as set forth in the following claims. Moreover, any document(s) mentioned herein are incorporated by reference in their entirety, as are any other documents that are referenced within the document(s) mentioned herein.

What is claimed is:

1. An orthopedic support device, comprising:
a main body formed of a first material having a predetermined elastic modulus and having at least one border area, the main body providing targeted compression and a substantially free range of movement to a body part when the orthopedic support device is worn so as to support the body part; and
at least one transition cuff attached to the main body and having a predetermined length, wherein the at least one transition cuff has a predetermined elastic modulus that is lower than the predetermined elastic modulus of the main body, and wherein the presence of the at least one transition cuff is effective to reduce displacement of soft tissue at the at least one border area that is caused by the targeted compression of the main body.

2. The orthopedic support device of claim 1, wherein the equation:
0.15(X)≦Y≦0.80(X)
is satisfied when the predetermined elastic modulus of the main body is X and the predetermined elastic modulus of the at least one transition cuff is Y.

3. The orthopedic support device of claim 1, wherein the predetermined length of the at least one transition cuff is about 1 inch.

4. The orthopedic support device of claim 1, wherein the orthopedic support device includes at least one of a proximal transition cuff and a distal transition cuff.

5. The orthopedic support device of claim 4, wherein the orthopedic support device includes a proximal transition cuff and a distal transition cuff, and wherein the proximal transition cuff has a first elastic modulus and the distal transition cuff has a second elastic modulus that is different than the first elastic modulus.

6. The orthopedic support device of claim 1, wherein the main body is made of a material selected from the group consisting of: a thermoplastic rubber, an elastic knit, an elastic weave, a silicone, a polyurethane, a polyvinylchloride, a styrene, an acrylic rubber, a butadiene, a choloroprene, a chlorosulfonated elastomer, an ethylene copolymer, an ethylene vinyl acetate, a fluoro-rubber, a natural rubber, a nitrile elastomer, a hi-nitrile phenolic rubber, an epicholorohydrin, and a vinyl plasticol.

7. The orthopedic support device of claim 1, wherein the at least one transition cuff is made of a material selected from the group consisting of: an elastic spacer fabric and a spandex.

8. The orthopedic support device of claim 1, wherein the at least one transition cuff has a top side and an underside, and wherein the at least one transition cuff includes at least one elastomeric attachment that is attached to the underside of the transition cuff.

9. The orthopedic support device of claim 1, wherein the main body has a uniform patterned shape of substantially constant thickness.

10. The orthopedic support device of claim 1, wherein the orthopedic support device is a sleeve type device.

11. The orthopedic support device of claim 1, wherein the orthopedic support device is a wrap type device

12. The orthopedic support device of claim 11, further comprising:
a first, proximal strap extending outwardly from the main body and having a first transition cuff attached thereto; and
a second, distal strap extending outwardly from the main body and having a second transition cuff attached thereto.

13. The orthopedic support device of claim 12, wherein the first, proximal strap extends outwardly from the main body in a different direction than the second, distal strap.

14. The orthopedic support device of claim 12, wherein first transition cuff is located along a distal end of the first, proximal strap, and wherein the second transition cuff is located along a proximal end of the second, distal strap.

15. A sleeve type orthopedic support device, comprising:
a main body formed of a resilient material having a predetermined elastic modulus and having at least one border area, the main body providing targeted compression and a substantially free range of movement to a body part when the orthopedic support device is worn so as to support the body part; and
at least one transition cuff attached to the main body and having a predetermined length, wherein the at least one transition cuff has a predetermined elastic modulus that is lower than the predetermined elastic modulus of the main body, and wherein the presence of the at least one transition cuff is effective to reduce displacement of soft tissue at the at least one border area that is caused by the targeted compression of the main body.

16. The orthopedic support device of claim 15, wherein the equation:
0.15(X)≦Y≦0.80(X)
is satisfied when the predetermined elastic modulus of the main body is X and the predetermined elastic modulus of the at least one transition cuff is Y.

17. A support brace for the knee comprising:
a substantially tubular sleeve, wherein the sleeve comprises:
a main body having a proximal end and a distal end and being made of a resilient material;
a transition cuff located at the proximal end of the main body and being made of an elastic spacer fabric,
wherein the main body of the sleeve has a first predetermined elastic modulus and the transition cuff of the sleeve has a second predetermined elastic modulus, and wherein the second predetermined elastic modulus is equal to about 15% to about 80% of the first predetermined elastic modulus.

18. The orthopedic support device of claim 17, wherein the resilient material is selected from the group consisting of: a knitted elastic and a neoprene laminate.

19. The orthopedic support device of claim 17, wherein the transition cuff has a length of about 1 inch.

20. The orthopedic support device of claim 17, wherein the transition cuff has a top side and an underside, and wherein the transition cuff includes at least one elastomeric attachment that is attached to the underside of the transition cuff.
